(19) United States
(12) Patent Application Publication
(10) Pub. No.: US 2011/0015650 A1
(43) Pub. Date: Jan. 20, 2011

(54) INSTRUMENT OF ROBOT ARM FOR SURGERY

(76) Inventors: Seung Wook Choi, Gyeonggi-do (KR); Jae Sun Lee, Gyeonggi-do (KR); Jong Seok Won, Gyeonggi-do (KR)

Correspondence Address: BIRCH STEWART KOLASCH & BIRCH, PO BOX 747, FALLS CHURCH, VA, 22040-0747, US

(21) Appl. No.: 12/922,630

(22) PCT Filed: Mar. 18, 2009
(86) PCT No.: PCT/KR09/01366

§ 371 Date: Sep. 14, 2010

(30) Foreign Application Priority Data
Jun. 11, 2008 (KR) 10-20008-0054474
Jun. 12, 2008 (KR) 10-2008-0055424

(51) Int. Cl. A61B 19/00

(52) U.S. Cl. 606/130

(57) ABSTRACT

An instrument for a surgical robot arm is disclosed. The instrument, which is to be mounted on a front end of a robot arm equipped with an actuator, includes: a housing, which is coupled to the front end of the robot arm; a driving wheel, which is coupled to the housing, and which is operated by way of a driving force transferred from the actuator; and a locking part, which is coupled to the housing, and which locks the operation of the driving wheel in correspondence to the mounting and dismounting of the housing on and from the robot arm. By installing a locking part on the instrument, the locking part can be made to restrain the rotation of the driving wheels when mounting or dismounting the instrument on or from the robot arm, and the driving wheels can be automatically calibrated when the instrument is dismounted from the robot arm. Thus, the driving wheels or the manipulation part may not undergo unnecessary movements, and the driving force of the robot arm can be transferred to the instrument without having to perform a separate aligning process after mounting the instrument onto the robot arm.

### CROSS-REFERENCE TO RELATED APPLICATIONS

This application is the National Phase of PCT/KR2009/001366 filed on Mar. 18, 2009, which claims priority under 35 U.S.C. 119(a) to Patent Application No. 10-2008-0054474 filed in the Republic of Korea on Jun. 11, 2008 and Patent Application No. 10-2008-0055424 filed in the Republic of Korea on Jun. 12, 2008, all of which are hereby expressly incorporated by reference into the present application.

### BACKGROUND

The present invention relates to an instrument of a robot arm for use in surgery.

In the field of medicine, surgery refers to a procedure in which a medical device is used to make a cut or an incision in or otherwise manipulate a patient's skin, mucosa, or other tissue, to treat a pathological condition. A surgical procedure such as a laparotomy, etc., in which the skin is cut open and an internal organ, etc., is treated, reconstructed, or excised, may entail problems of blood loss, side effects, pain, and scars, and as such, the use of robots is currently regarded as a popular alternative.

A set of surgical robots may include a master robot, which is manipulated by the doctor to generate and transmit the necessary signals, and a slave robot, which receives the signals from the master robot to actually apply the manipulation to the patient. The master robot and the slave robot can be arranged in the operating room as an integrated unit or as separate devices.

A slave robot may be equipped with a robot arm to make manipulations for surgery, while an instrument may be mounted on the front end of the robot arm. As illustrated in FIG. 1, a conventional instrument 54 may consist of a housing 108, a shaft 102 extending from the housing 108, and a pincer-like manipulation part 112 mounted on the far end 106 of the shaft 102 that is to be inserted into the surgical site. An interface part 110 may be formed on a bottom surface of the housing 108.

On a bottom surface of this type of conventional instrument 54, a multiple number of wheel-shaped drivers 118 may be coupled, as illustrated in FIG. 2. A wire connected to each portion of the manipulation part 112 may be wound respectively around a driver 118, so that when the driver 118 is rotated, a tensional force may be applied to the wire, causing the portion of the manipulation part 112 to move.

In order to mount an instrument 54 onto the robot arm, an adapter 128 such as that illustrated in FIG. 3 may be coupled to the front end of the robot arm. The adapter 128 may include guide fins for fitting on the interface part 110 of the housing 108, as well as actuators having shapes corresponding to the shapes of the drivers for transferring rotational forces to the drivers 118.

In this manner, a conventional instrument 54 may be mounted on the robot arm by sliding and fitting the housing 108 into the adapter 128. Then, the actuators equipped in the adapter 128 may rotate the drivers 118 to move the manipulation part 112 as necessary and conduct the surgery.

However, when the conventional instrument is mounted or dismounted from the robot arm, the drivers may have been rotated to different positions. As such, a calibrating process of aligning the actuators with the drivers may be required after mounting the instrument on the robot arm.

Also, when the instrument is dismounted from the robot arm, the rotation of the drivers may cause unnecessary movement of the manipulation part, or conversely, the movement of the manipulation part may cause the drivers to rotate to an undesired orientation, thus necessitating the above aligning process.

This process of aligning the instrument may unnecessarily consume time and effort during robot surgery procedures, and there is a risk that errors in the initial aligning process may lower the precision and reliability of the robot surgery.

Furthermore, for a conventional instrument, the shaft may be coupled to the side of the housing, while the drivers may be arranged on the bottom of the housing, so that when mounting the instrument onto the robot arm, the housing may have to be slided along at least the length of the bottom of the housing to be fitted onto the adapter. Therefore, an additional length of the shaft may be required tantamount to the length of the bottom of the housing.

Even in cases where the conventional instrument is mounted such that the interface part is put in direct contact with the actuators, instead of mounting the instrument by sliding the instrument along the direction in which the shaft is extended, there still remains a need for space around the robot arm for mounting and dismounting the housing. This problem may be more serious when replacing the instrument while the robot arm is close to the surgery patient. In some cases, the process of replacing the instrument may be obstructed by the surgery patient, possibly causing delays in surgery or even medical accidents.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention aims to provide a setup in which the instrument is automatically calibrated when mounted on or dismounted from the robot arm, such that it is unnecessary to perform an initial aligning process, and in which the driving wheels or the manipulation part do not undergo unnecessary movements when the instrument is dismounted from the robot arm.

Another aspect of the present invention is to provide a surgical instrument that can be mounted on a robot arm with a minimal amount of movement and does not require a separate space around the robot arm for mounting and dismounting the instrument.

Other technical problems addressed by the present invention will be readily understood from the descriptions that follow.

One aspect of the present invention provides an instrument for mounting on a front end of a robot arm equipped with an actuator. The instrument includes: a housing, which is coupled to the front end of the robot arm; a driving wheel, which is coupled to the housing, and which is operated by way of a driving force transferred from the actuator; and a locking part, which is coupled to the housing, and which locks the operation of the driving wheel in correspondence to the mounting and dismounting of the housing on and from the robot arm.

The locking part can lock the driving wheel when the housing is dismounted from the robot arm and unlock the driving wheel when the housing is mounted on the robot arm.

The instrument can further include a shaft that is coupled to the housing and a manipulation part that is mounted on a far end of the shaft and configured to move in correspondence with an operation of the driving wheel, where the locking part can lock the driving wheel in correspondence with the returning of the manipulation part to an initial position.

The locking part can include a switch that is activated in correspondence with a mounting and dismounting of the housing and a brake that restrains the rotation of the driving wheel in accordance to whether or not the switch is activated. A trigger can be formed on a front end of the robot arm that activates the switch in accordance to the mounting of the housing on the robot arm.

The switch can be coupled to the housing by way of an interposed elastic element, and the trigger can include a protrusion for pressing the switch. Here, the driving wheel can include a recess formed by a subsidence in a portion of the driving wheel, and the brake can be connected to the switch to be inserted into the recess in accordance to whether or not the switch is activated.

Also, the brake can be connected to the switch to clutch onto the driving wheel in accordance to whether or not the switch is activated. In this case, a recess can be formed in a surface of the driving wheel, and a protruding part corresponding with the recess can be formed on the brake.

The switch can include a sensor that generates a particular signal, while the trigger can include a contact that supplies electrical power to the sensor. In this case, the locking part can further include a control part, which receives signals from the sensor and generates a control signal corresponding with whether or not the brake is activated, and a motor, which receives the control signal to activate the brake.

Another aspect of the present invention provides a surgical instrument for mounting on a front end of a surgical robot arm equipped with an actuator. The surgical instrument includes: a shaft, which extends along a particular lengthwise direction; a housing, which is coupled to one end of the shaft, and which moves along the lengthwise direction to be joined to the front end of the robot arm; an interface part formed on that surface of the housing to which the shaft is coupled; and a driving wheel, which is coupled to the interface part, to operate by way of a driving force transferred from the actuator.

A manipulation part, which is to be inserted into a body of a surgery patient, can be coupled to the other end of the shaft, and the manipulation part can be made to move in correspondence with an operation of the driving wheel.

A sliding rail that extends along the lengthwise direction can be formed on the housing, while a guide rail can be formed in correspondence with the sliding rail on the front end of the robot arm. In this case, the front end of the robot arm can include a joining part that secures the housing onto the robot arm when the housing is moved such that the driving wheel is in contact with the actuator.

On the front end of the robot arm, a ledged part can be formed, on which the housing may rest facing the interface part. A hole or an indentation can be formed in the ledged part through which the shaft may be inserted. In this case, the actuator can be equipped on the ledged part facing the driving wheel.

The driving wheel can be shaped as a circular disc and can clutch onto the actuator such that the driving force is transferred to the driving wheel. For higher efficiency, a recess can be formed in a surface of the driving wheel, and a protruding part configured to be inserted in the recess can be formed on the actuator.

The driving wheel and/or the actuator can be supported by an elastic element that applies an elastic force in a direction which causes clutching between the driving wheel and the actuator.

Additional aspects, features, and advantages, other than those described above, will be obvious from the claims and written description below.

According to certain embodiments of the present invention as disclosed above, by installing a locking part on the instrument, the locking part can be made to restrain the rotation of the driving wheels when mounting or dismounting the instrument on or from the robot arm, and the driving wheels can be automatically calibrated when the instrument is dismounted from the robot arm. Thus, the driving wheels or the manipulation part may not undergo unnecessary movements, and the driving force of the robot arm can be transferred to the instrument without having to perform a separate aligning process after mounting the instrument onto the robot arm.

Also, in a surgical instrument, by forming the interface part and installing the driving wheels on the surface of the housing along the direction in which the shaft is extended, i.e. the direction in which the surgical instrument is mounted, the instrument can be mounted on the robot arm with a minimal amount of movement. Thus, it is not needed to unnecessarily increase the length of the shaft for mounting or dismounting the housing, and therefore the length of the instrument can be minimized.

Furthermore, since the instrument may be mounted and dismounted along the direction in which the shaft is extended, it is not needed to provide a separate space around the robot arm for mounting or dismounting the instrument, and therefore the size of the robot arm can be made compact. Thus, the robot arm may be positioned closer to the surgery patient, and the robot surgery may be performed with greater stability and higher reliability.

### BRIEF DESCRIPTION OF THE DRAWINGS

FIG. 1 through FIG. 3 illustrate a surgical instrument according to the related art.

FIG. 4 is a perspective view of an instrument according to an embodiment of the present invention.

FIG. 5 is a schematic drawing illustrating the operation of a locking part according to an embodiment of the present invention.

FIG. 6 is a plan view of a locking part according to an embodiment of the present invention.

FIG. 7 is a cross-sectional view across line A-A′ of FIG. 6.

FIG. 8 is a perspective view of a locking part according to an embodiment of the present invention.

FIG. 9 is a perspective view of a locking part according to another embodiment of the present invention.

FIG. 10 is a perspective view of a locking part according to another embodiment of the present invention.

FIG. 11 is a perspective view of a surgical instrument according to an embodiment of the present invention.

FIG. 12 is a perspective view of a surgical instrument and the front end of a robot arm according to an embodiment of the present invention.

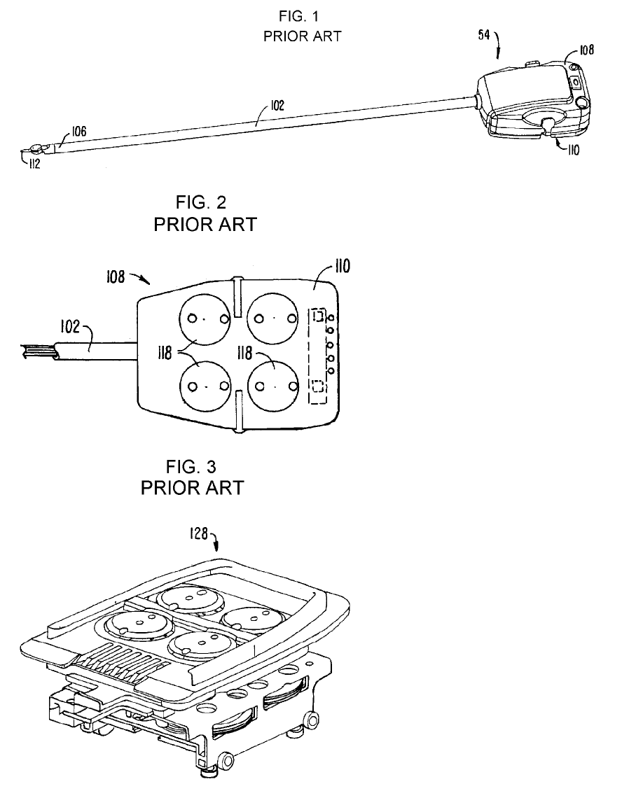

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the written description, certain detailed explanations of related art are omitted when it is deemed that they may unnecessarily obscure the essence of the present invention.

While such terms as “first” and “second,” etc., may be used to describe various components, such components must not be limited to the above terms. The above terms are used only to distinguish one component from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Certain embodiments of the present invention will be described below in detail with reference to the accompanying drawings. Those components that are the same or are in correspondence are rendered the same reference numeral regardless of the figure number, and redundant descriptions are omitted.

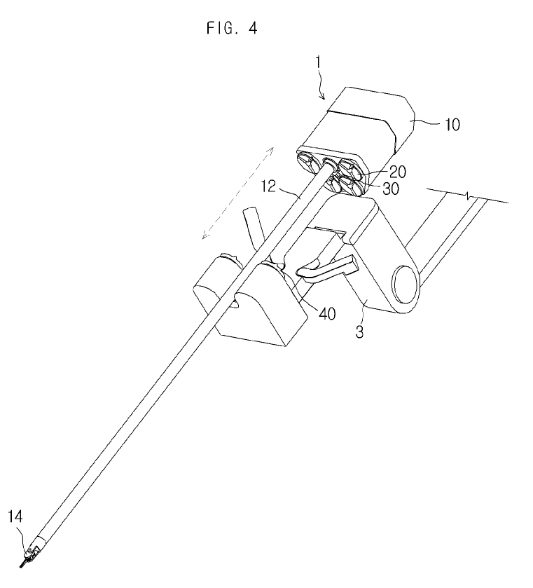

FIG. 4 is a perspective view of an instrument according to an embodiment of the present invention. Illustrated in FIG. 4 are an instrument 1, a robot arm 3, a housing 10, a shaft 12, a manipulation part 14, driving wheels 20, a locking part 30, and actuators 40.

A feature of this embodiment is that the driving wheels 20 are prevented from movement and locked automatically at the initial positions when the instrument 1 mounted on a surgical robot arm 3 is dismounted from the robot arm 3, so that the instrument 1 may be used immediately without requiring an initial alignment process when the instrument 1 is mounted again on the robot arm 3.

An instrument 1 according to this embodiment may be mounted on a front end of a surgical robot arm 3, where actuators 40 for transferring the driving forces to the instrument 1 may be equipped on the front end of the robot arm 3. The instrument 1 may be composed mainly of a housing 10, a shaft 12 that extends from the housing 10, and a manipulation part 14 coupled to a far end of the shaft 12.

The instrument 1 may be mounted on the front end of the robot arm 3 which is formed in a shape corresponding with the shape of the housing 10 of the instrument 1. A surface of the housing 10 according to this embodiment can serve as an interface part, and correspondingly, a hook, guide, detent, etc., can be formed on the front end of the robot arm 3 for coupling with the interface part.

When the instrument 1 is mounted on the front end of the robot arm 3, the driving forces may be transferred from the robot arm 3 through the actuators 40 to the driving wheels 20 coupled to the housing 10. Wires may be wound around the driving wheels 20, and these wires may be connected through the shaft 12 to respective portions of the manipulation part 14 coupled to the far end. Thus, as the driving wheels 20 are rotated by the driving forces transferred from the robot arm 3, the tensional forces in the wires cause the respective portions of the manipulation part 14 to move, making it possible to manipulate the instrument 1 by way of the surgical robot.

As the actuators 40 may serve to transfer the driving forces to the driving wheels 20, various power-transferring mechanisms, such as wheels, sliders, gears, etc., having structures corresponding with the shapes of the driving wheels 20 can be used for the actuators 40.

A locking part 30 may be coupled onto the housing 10 of an instrument 1 according to the present embodiment. The locking part 30 may serve to lock the operation of the driving wheels 20 such that the driving wheels 20 remain unmoved and secured at their initial positions. In other words, when the instrument 1 is mounted on the robot arm 3, the driving wheels 20 may be operated by the actuators 40, but when the instrument 1 is dismounted from the robot arm 3, the driving wheels 20 may be secured and remain unmoved.

If, while the instrument 1 is not mounted on the robot arm 3, the manipulation part 14 can be moved by the rotation of the driving wheels 20, and conversely, the driving wheels 20 can be rotated by moving the manipulation part 14, then the actuators 40 and the driving wheels 20 may not be aligned, when the instrument 1 is mounted on the robot arm 3 after the driving wheels 20 have been randomly rotated. This can cause the surgical robot to malfunction.

By installing the locking part 30 on the instrument 1, as in the present embodiment, the driving wheels 20 can be prevented from moving when the instrument 1 is not mounted on the robot arm 3, and hence the manipulation part 14 may also be locked in an immovable state. By thus having the instrument 1 locked in position, the instrument 1 can be prevented from undergoing unnecessary movements while dismounted from the robot arm 3.

Furthermore, if the driving wheels 20 are secured such that the instrument 1 is in its initial position, i.e. the position in which the instrument 1 is mounted on the robot arm 3 and the manipulation part 14 is inserted into the body of the surgery patient, then the actuators 40 and the driving wheels 20 can be aligned with each other as soon as the instrument 1 is mounted on the robot arm 3. Thus, it is possible to use the instrument 1 immediately after mounting the instrument 1 on the robot arm 3, without having to perform a separate aligning process, such as of idly rotating the actuators 40.

The operation and composition of a locking part 30 according to the present embodiment will be described below in more detail.

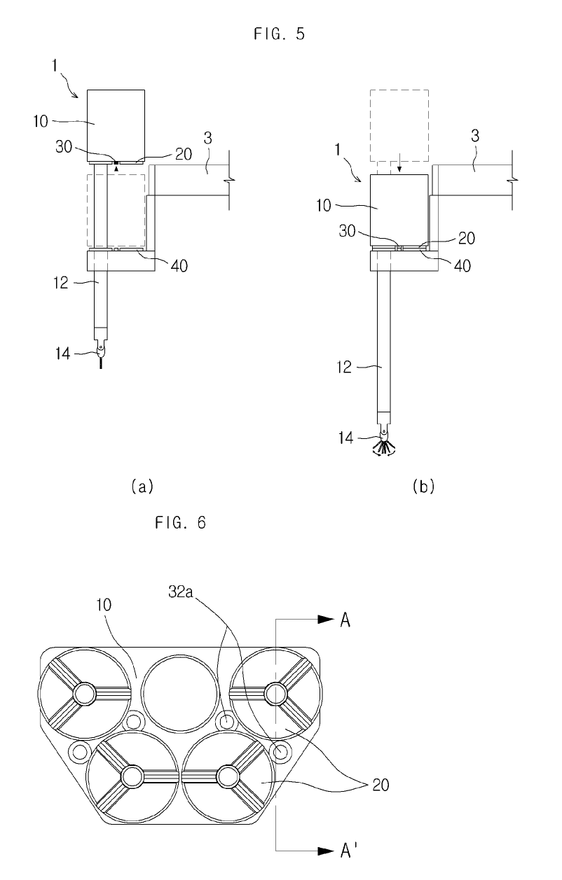

FIG. 5 is a schematic drawing illustrating the operation of a locking part 30 according to an embodiment of the present invention. Illustrated in FIG. 5 are an instrument 1, a robot arm 3, a housing 10, a shaft 12, a manipulation part 14, driving wheels 20, a locking part 30, and actuators 40.

The locking part 30 according to this embodiment allows the driving wheels 20 to move in accordance to the actuators 40, when the instrument 1 is mounted on the robot arm 3, but secures the driving wheels 20, when the instrument 1 is dismounted from the robot arm 3.

When the housing 10 of the instrument 1 is dismounted from the robot arm 3, as in illustration (a) of FIG. 5, the locking part 30 according to the present embodiment may operate correspondingly to lock the driving wheels 20 such that the driving wheels 20 are unable to move. Thus, the instrument 1 may be prevented from unnecessary movement, and later when the instrument 1 is again mounted on the robot arm 3, the driving wheels 20 and the actuators 40 may be aligned immediately.

Also, when the housing 10 of the instrument 1 is mounted on the robot arm 3, as in illustration (b) of FIG. 5, the locking part 30 according to the present embodiment may operate correspondingly to unlock the driving wheels 20 such that the driving wheels 20 are able to move freely. Thus, the driving wheels 20 aligned with the actuators 40 of the robot arm 3 may be operated by driving the actuators 40, and the manipulation part 14 of the instrument 1 may be moved to a desired position, as described above.

During robot surgery, the instrument 1 may be mounted on the robot arm 3 in an initial position, and the manipulation part 14 on the far end of the instrument 1 may be inserted into the body of the surgery patient. When the surgery is finished, or when the instrument 1 is to be replaced, the manipulation part 14 may be returned to its initial position before withdrawing from the surgery patient.

Here, the “initial position” can refer to the position before the manipulation part 14 was moved, i.e. a position facing a direction parallel to the shaft 12 with the pincers closed, rather than a position in which the manipulation part 14 is rotated to face a particular direction or the pincers are opened for surgery. By having the manipulation part 14 return to its initial position, the instrument 1 can be inserted unbothered into the surgical site and can be withdrawn from the surgical site without damaging other organs.

As such, for a locking part 30 according to this embodiment, it may be desirable to lock the operation of the driving wheels 20 after the manipulation part 14 has returned to its initial position. This is because the occasions for mounting or dismounting the instrument 1 on or from robot arm 3 are when the manipulation part 14 is inserted into or withdrawn from the surgical site.

In other words, when mounting the instrument 1 on the robot arm 3, it may be more efficient to have the driving wheels 20 aligned with the actuators 40 at the positions assumed by the driving wheels 20 when the manipulation part 14 is in an initial position. Accordingly, when dismounting the instrument 1 from the robot arm 3, it may be efficient for later use to have the manipulation part 14 return to its initial position, so that the driving wheels 20 and actuators 40 may also return to their initial positions.

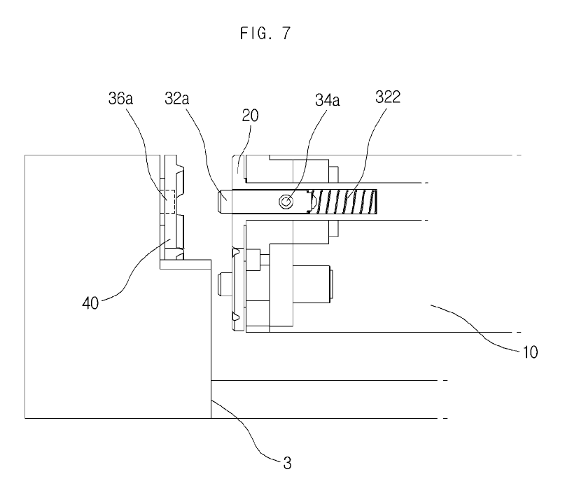

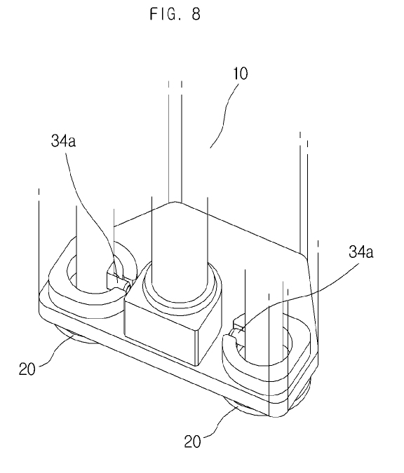

FIG. 6 is a plan view of a locking part according to an embodiment of the present invention, FIG. 7 is a cross-sectional view across line A-A′ of FIG. 6, and FIG. 8 is a perspective view of a locking part according to an embodiment of the present invention. Illustrated in FIG. 6 through FIG. 8 are a robot arm 3, a housing 10, driving wheels 20, switches 32a, brakes 34a, triggers 36a, actuators 40, and elastic elements 322.

A locking part 30 according to the present embodiment can include switches 32a and brakes 34a, which secure the driving wheels 20 according to the activation of the switches 32a such that the driving wheels 20 are prevented from rotating. The switches 32a may be activated in correspondence with the mounting or dismounting of the housing 10 of the instrument 1 on or from the robot arm 3.

To enable the switches 32a to activate according to the mounting or dismounting of the housing 10, triggers 36a may be formed on the robot arm 3 that correspond with the switches 32a. Thus, when the instrument 1 is mounted on the robot arm 3, the triggers 36a may activate the switches 32a, and the brakes 34a may lock or unlock the driving wheels 20 according to the activation of the switches 32a.

The embodiment illustrated in FIG. 6 to FIG. 8 shows one example in which the locking part 30 described above can be implemented as a mechanical device, where the locking part 30 may be operated in correspondence with the mounting and dismounting of the instrument 1 on or from the robot arm 3 without requiring a separate power source.

In this embodiment, a portion of a driving wheel 20 may be subsided to form a recess, and a brake 34a may be formed in the shape of a detent pin that can be inserted into the recess. A switch 32a may be shaped as a movable pin that is connected to the brake 34a and exposed at one side of the housing 10. The switch 32a may be supported on the housing 10 by an elastic element 322, and a portion on the one side of the housing 10 may be perforated, to expose the switch 32a. A trigger 36a may be formed as a protrusion erected on the robot arm 3 in correspondence with the position of the switch 32a.

Looking at the operation of the locking part 30 mechanically implemented as above, when the housing 10 is mounted on the robot arm 3, the triggers 36a may press the switches 32a, at which the brakes 34a connected to the switches 32a, i.e. the movable pins, may be removed from the recesses of the driving wheels 20. As the brakes 34a, i.e. detent pins, which were inserted in the recesses, are removed from the recesses, the driving wheels 20 are able to rotate freely, and the driving forces transferred from the actuators 40 of the robot arm 3 may rotate the driving wheels 20.

When the housing 10 is dismounted from the robot arm 3, the restoring forces of the elastic elements 322 that support the switches 32a may return the switches 32a to their original positions, so that the brakes 34a connected to the switches 32a may again be inserted into the recesses of the driving wheels 20. When the brakes 34a are inserted into the recesses in this manner, the brakes 34a may restrain the rotation of the driving wheels 20, whereby the driving wheels 20 may be secured and immovable. Thus, the driving wheels 20 may be locked.

The shapes and structures of the switches 32a, triggers 36a, and brakes 34a according to the present embodiment are not necessarily limited to the shapes of movable pins supported by elastic element 322 and detent pins connected to the movable pins, as in the illustrations of FIG. 6 to FIG. 8. Obviously, various mechanical compositions may be applied that are capable of locking or unlocking the driving wheels 20 in correspondence to the mounting or dismounting of the housing 10.

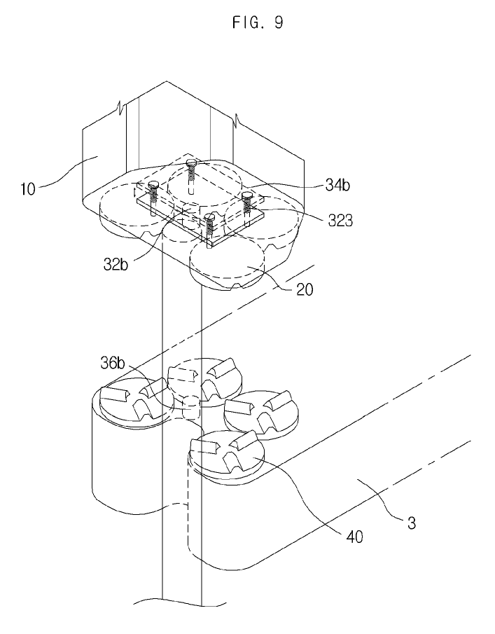

A description will now be provided, with reference to FIG. 9, on another embodiment illustrating a mechanical implementation of the locking part.

FIG. 9 is a perspective view of a locking part according to another embodiment of the present invention. Illustrated in FIG. 9 are a robot arm 3, a housing 10, driving wheels 20, a switch 32b, a brake 34b, a trigger 36b, actuators 40, and elastic elements 323.

Similar to the previously described embodiment, the locking part 30 according to this embodiment can also include a switch 32b and a brake 34b that secure the driving wheels 20 according to the activation of the switch 32b such that the driving wheels 20 are prevented from rotating. The switch 32b may be activated in correspondence with the mounting or dismounting of the housing 10 of the instrument 1 on or from the robot arm 3.

To enable the switch 32b to activate according to the mounting or dismounting of the housing 10, a trigger 36b may be formed on the robot arm 3 in correspondence with the switch 32b. Thus, when the instrument 1 is mounted on the robot arm 3, the trigger 36b may activate the switch 32b, and the brake 34b may lock or unlock the driving wheels 20 according to the activation of the switch 32b.

The embodiment illustrated in FIG. 9 shows another example in which the locking part 30 described above can be implemented as a mechanical device, where the locking part 30 may be operated in correspondence with the mounting and dismounting of the instrument 1 on or from the robot arm 3 without requiring a separate power source.

In this embodiment, the brake 34b may be formed as a friction plate, while the switch 32b may be formed as a leg that is connected to the brake 34b and exposed at one side of the housing 10. The switch 32b may be coupled to the housing 10 with elastic elements 323 positioned in-between, and a portion on the one side of the housing 10 may be perforated, to expose the switch 32b. The trigger 36b may be formed as a protrusion erected on the robot arm 3 in correspondence with the position of the switch 32b.

Looking at the operation of the locking part 30 mechanically implemented as above, when the housing 10 is mounted on the robot arm 3, the trigger 36b may press the switch 32b, at which the brake 34b connected to the switch 32b may be separated from the driving wheels 20. As the brake 34b, which was placed in contact with the driving wheels 20, is separated from the driving wheels 20, the driving wheels 20 are able to rotate freely, and the driving forces transferred from the actuators 40 of the robot arm 3 may rotate the driving wheels 20.

When the housing 10 is dismounted from the robot arm 3, the restoring forces of the elastic elements 322 interposed between the switch 32b and the housing 10 may return the switch 32b to its original position, so that the brake 34b connected to the switch 32b may again be placed in contact with the driving wheels 20. When the brake 34b clutches onto the driving wheels 20 in this manner, the brake 34b may apply friction against the rotation of the driving wheels 20, and with a sufficient amount of resistance, the driving wheels 20 may be secured and made immovable. Thus, the driving wheels 20 may be locked.

Here, the locking of the driving wheels 20 may depend on the resistive force resulting from the friction between the brake 34b and the driving wheels 20. To ensure that a sufficient resistive force is applied by the brake 34b, the restorative force of the elastic elements 323 or the surface roughness of the brake 34b may be increased.

Furthermore, as illustrated in FIG. 9, it is possible to form protruding parts on the surface of the brake 34b and form corresponding recesses in the surfaces of the driving wheels 20 in which the protruding parts may be inserted. By thus processing the surfaces of the driving wheels 20 and the brake 34b, the brake 34b can be made to apply resistive forces required for the driving wheels 20, even if the restorative forces of the elastic elements 323 or the surface roughness of the brake 34b are not sufficient great.

The shapes and structures of the switch 32b, trigger 36b, and brake 34b according to the present embodiment are not necessarily limited to those illustrated in FIG. 9. Obviously, various mechanical compositions may be applied that are capable of locking or unlocking the driving wheels 20 in correspondence to the mounting or dismounting of the housing 10.

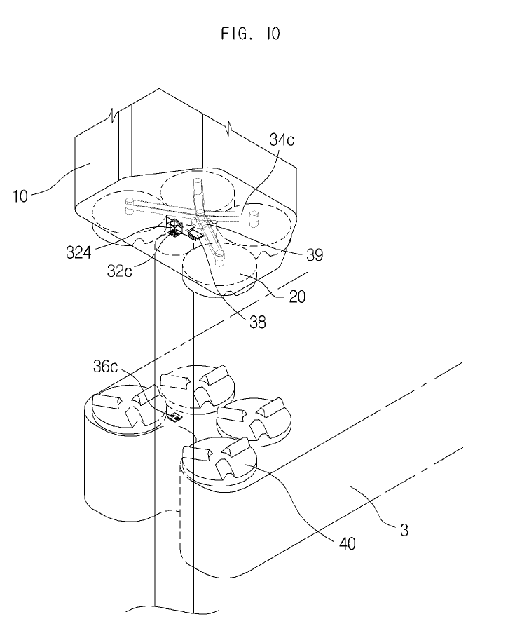

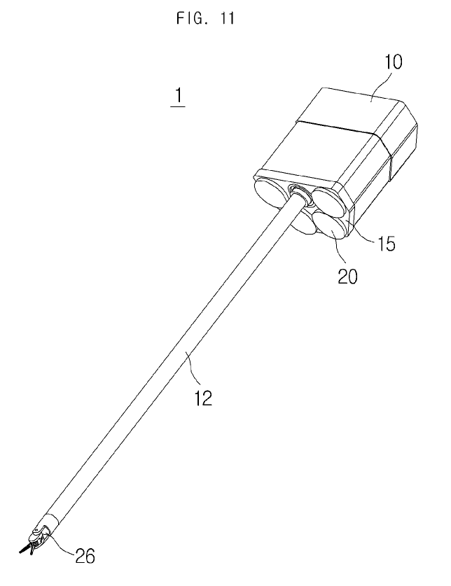

FIG. 10 is a perspective view of a locking part according to another embodiment of the present invention. Illustrated in FIG. 10 are a robot arm 3, a housing 10, driving wheels 20, a switch 32c, brakes 34c, a trigger 36c, a control part 38, a motor 39, actuators 40, and a sensor 324.

The embodiment illustrated in FIG. 9 shows an example in which the locking part 30 described above can be implemented as an electrical device, where an action of mounting or dismounting the instrument 1 on or from the robot arm 3 may be detected, and the locking part 30 may be operated according to a corresponding signal using a separate power source. The basic functions of the major components of the locking part 30, namely, the switch, brakes, and trigger, can be substantially the same as those in the previously described embodiments, and therefore will not be described in excessive detail.

In this embodiment, the switch 32c may include a sensor 324 that generates signals in correspondence to the mounting and dismounting actions of the housing 10, and the brakes 34c may be formed as belts that are wound around the shafts of the driving wheels 20 and activated by a separate power source. A portion of the sensor 324 included in the switch 32c may be exposed at one side of the housing 10. The trigger 36c may be formed as an electrical contact formed on the robot arm 3 in correspondence with the position of the switch 32c, and electrical power can be supplied to the sensor 324 via this contact.

Looking at the operation of the locking part 30 electrically implemented as above, when the housing 10 is mounted on the robot arm 3, the triggers 36c and the sensor 324 may be electrically connected, at which the switch 32c may generate a particular signal (hereinafter referred to as a “mount signal”). As the mount signal is transferred to the brakes 34c, the belts wound around the driving wheels 20 may be loosened, allowing the driving wheels 20 to rotate freely, and enabling the driving forces transferred from the actuators 40 of the robot arm 3 to rotate the driving wheels 20.

When the housing 10 is dismounted from the robot arm 3, the electrical connection between the trigger 36c and the sensor 324 may be disconnected, at which the switch 32c may generate a particular signal (hereinafter referred to as a “dismount signal”). As the dismount signal is transferred to the brakes 34c, the belts wound around the driving wheels 20 may be tightened, applying friction to the rotation of the driving wheels 20, and with a sufficient amount of resistance, the driving wheels 20 may be secured and made immovable. Thus, the driving wheels 20 may be locked.

Here, a separate control part 38, such as a microprocessor, etc., may additionally be included for receiving the signals from the switch 32c and controlling the activation of the brakes 34c. The control part 38 may serve to receive the mount signal or dismount signal from the sensor 324 of the switch 32c, determine whether to tighten or loosen the belts on the brakes 34c, and transfer a corresponding control signal to the brakes 34c.

In addition, the brakes 34c can be connected to a motor 39 and can be made to receive signals from the control part 38, where the motor 39 may receive the control signals to loosen the belts of the brakes 34c or tighten the belts of the brakes 34c to provide sufficient resistance to secure the driving wheels 20.

The shapes and structures of the switch 32c, trigger 36c, brakes 34c, control part 38, and motor 39 according to the present embodiment are not necessarily limited to those illustrated in FIG. 10. Obviously, various electrical compositions may be applied that are capable of locking or unlocking the driving wheels 20 in correspondence to the mounting or dismounting of the housing 10.

FIG. 11 is a perspective view of a surgical instrument according to an embodiment of the present invention. Illustrated in FIG. 11 are an instrument 1, a housing 10, a shaft 12, an interface part 15, driving wheels 20, and a manipulation part 26.

A feature of this embodiment is that the driving wheels 20 of the instrument 1, which is to be mounted on a surgical robot arm, is formed on the bottom surface along the direction in which the instrument 1 is mounted, and likewise the actuator is formed in a corresponding position on the robot arm. Thus, the length of the instrument 1 can be shortened, and the space required for mounting and dismounting the instrument 1 can be minimized.

The basic structure of an instrument 1 according to the present embodiment may include a housing 10, a shaft 12 extending from the housing 10, and a manipulation part 26 coupled to the end of the shaft 12. Defining the “lengthwise direction” as the direction in which the shaft 12 extends from the instrument 1, the instrument 1 according to the present embodiment may be mounted along the lengthwise direction, and for this purpose, an interface part 15 may be formed on the housing 10 facing the lengthwise direction.

That is, on the housing 10 of an instrument 1 according to this embodiment, the interface part 15 may be formed in the lengthwise direction, which is the direction in which the instrument 1 is mounted on the robot arm. The driving forces and other required signals may be received from the robot arm via the interface part 15.

For this purpose, the interface part 15 may be formed on the housing 10 along the lengthwise direction, and driving wheels 20 may be arranged on the interface part 15, as illustrated in FIG. 11. The manipulation part 26 of the instrument 1 illustrated in FIG. 11 may be manipulated with 4 degrees of freedom and thus may be equipped with four driving wheels 20, but it is not imperative that four driving wheels 20 be installed, and it is obvious that a greater number or a lower number of driving wheels 20 can be arranged as necessary to move the manipulation part 26.

The instrument 1 according to this embodiment may be mounted on the front end of a robot arm, which may be formed in a shape corresponding with that of the housing 10. As described above, the interface part 15 may be formed in the direction in which the housing 10 is mounted (the lengthwise direction), and the driving wheels 20 may be arranged on the interface part 15. In the front end of the robot arm, guide rails may be formed that correspond with the driving wheels 20 and allow the housing 10 to be fitted on, and a joining part may be included for securing the mounted housing 10. The guide rails and the joining part will be described later in further detail.

When the instrument 1 is mounted on the robot arm 3, the driving forces may be transferred to the driving wheels 20 of the housing 10 via actuators equipped on the front end of the robot arm 3. In the descriptions that follow, the components that transfer driving forces from the robot arm to the instrument will be referred to as “actuators.” As the actuators 40 may serve to transfer the driving forces to each of the multiple driving wheels 20, various power-transferring mechanisms, such as wheels, sliders, gears, etc., that correspond with the driving wheels 20 can be used for the actuators 40.

Wires may be wound around the multiple number of driving wheels 20, and these wires may be connected through the shaft 12 to respective portions of the manipulation part 26 coupled to the far end. Thus, as the driving wheels 20 are rotated by the driving forces transferred from the robot arm, the tensional forces in the wires cause the respective portions of the manipulation part 26 to move, making it possible to manipulate the instrument 1 by way of the surgical robot.

According to this embodiment, the instrument 1 may be structured to be joined to the robot arm as the housing 10 is moved along the lengthwise direction. For this purpose, the interface part 15 may be formed on the side of the housing facing the lengthwise direction, i.e. the side of the housing 10 to which the shaft 12 is coupled. The interface part 15 may serve as a medium for transferring the driving forces and other signals between the instrument 1 and the robot arm.

Thus, when the housing 10 is mounted on the robot arm, the interface part 15 may be placed in contact with the surface of the robot arm on which the actuators are formed. By installing the driving wheels 20 on the interface part 15, as illustrated in FIG. 11, the driving wheels 20 can be placed in contact with the actuators, so that the driving wheels 20 may receive the driving forces transferred via the actuators and thereby operate. Here, to place the driving wheels 20 in contact with the actuators is to mount the instrument 1 on the robot arm, whereas to separate the driving wheels 20 from the actuators is to dismount the instrument 1 from the robot arm. As such, the instrument 1 can be mounted or dismounted on or from the robot arm with only a minimal amount of movement.

According to this embodiment, the extra length of the shaft 12 required for mounting and dismounting the instrument 1 is almost zero, and the length of the shaft 12 need not be unnecessarily increased. As a result, the robot arm may be manipulated at a position closer to the surgery patient, and the robot surgery may be performed with greater stability and reliability.

Also, since the instrument 1 may be mounted or dismounted on or from the robot arm by moving the housing 10 along the lengthwise direction, it is not needed to provide a separate space around the robot arm for mounting and dismounting the instrument 1, and the surgical robot can be designed with a more compact size.

The manipulation part 26 may be installed on the other end of the shaft 12, and in the case where the manipulation part 26 is shaped as a pair of pincers, for example, each portion may be respectively connected by a wire, etc., to a driving wheel 20, so that the manipulation part 26 may be made to perform rotating or grabbing actions by operating the driving wheels 20. Thus, during a robot surgery procedure, the manipulation part 26 installed on the far end of the shaft 12 may be inserted into the body of the surgery patient to perform the actions required for surgery.

One feature of the instrument 1 according to the present embodiment is that the instrument 1 may be mounted on the robot arm by moving along the lengthwise direction. As such, a sliding rail that extends along the lengthwise direction can be formed on the housing 10. The sliding rail can be formed in a variety of shapes, such as troughs, trenches, grooves, protrusions, and rails formed on the housing 10 along the lengthwise direction, which enable the instrument 1 to be mounted on the robot arm by moving along the lengthwise direction.

If a sliding rail is formed on the housing 10, a guide rail corresponding with the sliding rail can be formed on the front end of the robot arm. If the sliding rail is shaped as a trough, trench, or groove, the guide rail can be shaped as a protrusion that may be inserted in the slide rail, and if the sliding rail is shaped as a protrusion or a rail, the guide rail can be shaped as a trench in which the slide rail may be inserted.

Of course, various other mating structures formed in the lengthwise direction can be used as the sliding rail and guide rail according to the present embodiment.

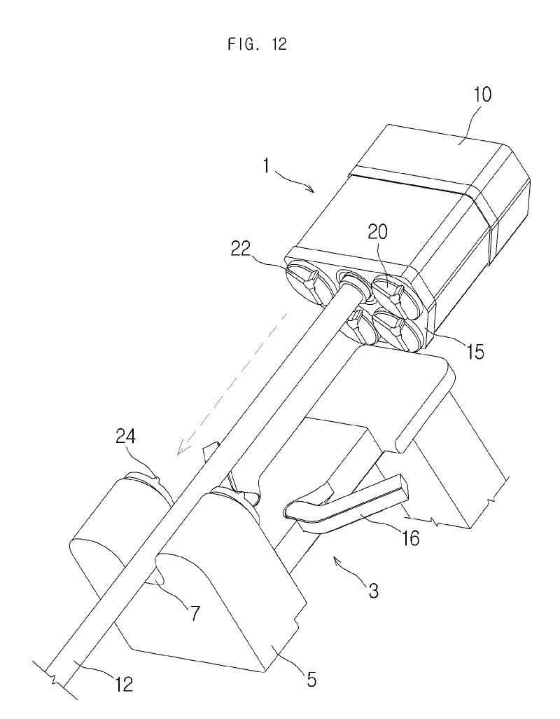

FIG. 12 is a perspective view of a surgical instrument and the front end of a robot arm according to an embodiment of the present invention. Illustrated in FIG. 12 are an instrument 1, a robot arm 3, a ledged part 5, an indentation 7, a housing 10, a shaft 12, an interface part 15, a joining part 16, driving wheels 20, actuators 40, recesses 22, and protruding parts 24.

An instrument 1 according to this embodiment can be mounted on the front end of a robot arm 3 by moving the housing 10 along the lengthwise direction. In relation to this, a joining part 16 for securing the mounted housing 10 can be equipped on the front end of the robot arm 3.

That is, after moving the housing 10 along the lengthwise direction and mounting the housing 10 on the robot arm 3, the housing 10 may have to be secured to the robot arm 3 in order to proceed with robot surgery. As such, a component may be included for securing the housing 10 to the front end of the robot arm 3. Various mechanisms such as stoppers, hooks, levers, etc., can be employed for the joining part 16. FIG. 11 illustrates an example in which a pair of levers are used for the joining part 16.

In this case, the housing 10 may be moved along the lengthwise direction such that the driving wheels 20 installed on the interface part 15 are placed in contact with the actuators 40 of the robot arm 3, after which the pair of levers may be operated to secure the housing 10 to the robot arm 3. When dismounting the housing 10 from the robot arm 3, the levers may be operated in reverse to release the housing 10.

By forming hooks, etc., at the ends of the levers, the levers can be made to operate automatically according to the movement of the housing 10, so that the levers may be engaged when the interface part 15 contacts the actuators 40 to automatically secure the housing 10. Of course, various other structures for securing the housing 10 to the robot arm 3 can be used for the joining part 16.

The front end of a robot arm 3 according to this embodiment can be formed in a shape corresponding with that of the instrument 1. That is, for the purpose of joining the instrument 1 to the robot arm 3 by moving the housing 10 along the lengthwise direction, a ledged part 5 on which to rest the housing 10 can be formed on the robot arm 3. Since a shaft 12 extending along the lengthwise direction is coupled to the housing 10, a hole or an indentation 7 through which the shaft 12 may pass can be formed in the ledged part 5, as illustrated in FIG. 12, to allow the housing 10 to rest on the ledged part 5.

In mounting the instrument 1, the shaft 12 may be placed through the hole or indentation 7 formed in the ledged part 5, enabling the housing 10 to rest on the ledged part 5 without interference from the shaft 12.

As the ledged part 5 is where the housing 10 is to be rested, mounting the instrument 1 may result in the housing 10 resting on the ledged part 5 and the interface part 15 touching a surface of the ledged part 5. Thus, by installing the actuators 40 on the surface of the ledged part 5 of the robot arm 3 that is placed in contact with the interface part 15, the actuators 40 can be aligned with the driving wheels 20. In other words, by installing the actuators 40 on the ledged part 5 opposite the positions of the driving wheels 20, the driving wheels 20 can be aligned with the actuators 40 as soon as the interface is placed in contact with the ledged part 5.

In cases where the actuators 40 are formed as rotating circular discs, if the driving wheels 20 are formed as circular discs that touch the actuators 40, the driving wheels 20 can be made to clutch onto the actuators 40 when the driving wheels 20 are placed in contact with the actuators 40, whereby the driving forces can be transferred from the actuators 40 to the driving wheels 20.

In order to increase the efficiency of the transfer of driving forces from the actuators 40 to the driving wheels 20, recesses 22 can be formed in the surfaces of the driving wheels 20, and protruding parts 24 that may be inserted in the recesses 22 can be formed in the surfaces of the actuators 40, as illustrated in FIG. 12. By thus forming the recesses 22 and protruding parts 24, the driving wheels 20 may be prevented from idle rotation, and the rotational forces of the actuators 40 may be transferred directly to the driving wheels 20, when the driving wheels 20 clutch onto the actuators 40.

The actuators 40 according to the present embodiment can be coupled to the robot arm 3 by way of elastic elements (not shown), such as springs, etc., positioned in-between. That is, the elastic elements such as springs, etc., which support the actuators 40 may serve as what is known as “spring cushions,” so that the actuators 40 and the driving wheels 20 may be clutched more firmly. By mounting spring cushions on the actuators 40 in this manner, improper engaging between the actuators and the driving wheels 20 (backlash), which may result in improper transfer of driving forces from the actuators 40 to the driving wheels 20, can be prevented. The spring cushions can be mounted not only on the actuators 40 but also on the driving wheels 20.

Also, if spring cushions are positioned on the actuators 40 and/or the driving wheels 20, the “calibrating” process, which may entail rotating the actuators 40 for alignment if the protruding parts 24 of the actuators 40 are not aligned with the recesses 22 of the driving wheels 20 during the mounting of the instrument 1, can be performed more easily and with less damage to the actuators 40 and/or driving wheels 20.

However, it is not imperative that recesses 22 be formed in the driving wheels 20 and protruding parts 24 be formed in the actuators 40 to increase the efficiency of the transfer of driving forces. It is obvious that the protruding parts 24 can be formed on the driving wheels 20, while the recesses 22 can be formed in the actuators 40, and that various other methods can be applied for increasing the efficiency of the transfer of driving forces in the clutching mechanism, such as by increasing the surface roughness of the driving wheels 20 and actuators 40.

While the present invention has been described with reference to particular embodiments, it will be appreciated by those skilled in the art that various changes and modifications can be made without departing from the spirit and scope of the present invention, as defined by the claims appended below.

What is claimed is:

1. An instrument for mounting on a front end of a robot arm equipped with an actuator, the instrument comprising:
a housing coupled to the front end of the robot arm;
a driving wheel coupled to the housing, the driving wheel configured to operate by way of a driving force transferred from the actuator; and
a locking part coupled to the housing, the locking part configured to lock an operation of the driving wheel in correspondence to a mounting and dismounting of the housing on and from the robot arm.

2. The instrument of claim 1, wherein the locking part is configured to lock the driving wheel when the housing is dismounted from the robot arm.

3. The instrument of claim 2, wherein the locking part unlocks the driving wheel when the housing is mounted on the robot arm.

4. The instrument of claim 1, further comprising:
a shaft coupled to the housing; and
a manipulation part mounted on a far end of the shaft, the manipulation part configured to move in correspondence with an operation of the driving wheel,
wherein the locking part is configured to lock the driving wheel in correspondence with a returning of the manipulation part to an initial position.

5. The instrument of claim 1, wherein the locking part comprises:
a switch configured to activate in correspondence with a mounting and dismounting of the housing; and
a brake configured to restrain a rotation of the driving wheel in accordance to whether or not the switch is activated.

6. The instrument of claim 5, wherein a trigger is formed on a front end of the robot arm, the trigger configured to activate the switch in accordance to a mounting of the housing on the robot arm.

7. The instrument of claim 6, wherein the switch is coupled to the housing by way of an interposed elastic element, and the trigger comprises a protrusion configured to press the switch.

8. The instrument of claim 7, wherein the driving wheel comprises a recess, the recess formed by a subsidence in a portion of the driving wheel, and
the brake is connected to the switch and is configured to be inserted into the recess in accordance to whether or not the switch is activated.

9. The instrument of claim 7, wherein the brake is connected to the switch and is configured to clutch onto the driving wheel in accordance to whether or not the switch is activated.

10. The instrument of claim 9, wherein a recess is formed in a surface of the driving wheel, and a protruding part corresponding with the recess is formed on the brake.

11. The instrument of claim 6, wherein the switch comprises a sensor configured to generate a particular signal, and the trigger comprises a contact configured to supply electrical power to the sensor.

12. The instrument of claim 11, further comprising:
a control part configured to receive signals from the sensor and generate a control signal corresponding with whether or not the brake is activated; and
a motor configured to receive the control signal and activate the brake.

13. A surgical instrument for mounting on a front end of a surgical robot arm equipped with an actuator, the surgical instrument comprising:
a shaft extending along a particular lengthwise direction;
a housing coupled to one end of the shaft, the housing configured to move along the lengthwise direction to be joined to the front end of the robot arm;
an interface part formed on that surface of the housing to which the shaft is coupled; and
a driving wheel coupled to the interface part, the driving wheel configured to operate by way of a driving force transferred from the actuator.

14. The surgical instrument of claim 13, wherein a manipulation part is coupled to the other end of the shaft, the manipulation part to be inserted into a body of a surgery patient, and
the manipulation part is configured to move in correspondence with an operation of the driving wheel.

15. The surgical instrument of claim 13, wherein a sliding rail extending along the lengthwise direction is formed on the housing, and
a guide rail is formed on the front end of the robot arm in correspondence with the sliding rail.

16. The surgical instrument of claim 15, wherein the front end of the robot arm comprises a joining part configured to secure the housing onto the robot arm when the housing is moved such that the driving wheel is in contact with the actuator.

17. The surgical instrument of claim 13, wherein a ledged part is formed on the front end of the robot arm, the ledged part configured to have the housing rested thereon facing the interface part, and
the ledged part includes a hole or an indentation formed therein through which the shaft is inserted.

18. The surgical instrument of claim 17, wherein the actuator is equipped on the ledged part facing the driving wheel.

19. The surgical instrument of claim 13, wherein the driving wheel is shaped as a circular disc and is configured to clutch onto the actuator such that the driving force is transferred to the driving wheel.

20. The surgical instrument of claim 19, wherein a recess is formed in a surface of the driving wheel, and a protruding part configured to be inserted in the recess is formed on the actuator.

21. The surgical instrument of claim 19, wherein at least one of the driving wheel and the actuator is supported by an elastic element configured to apply an elastic force in a direction which causes clutching between the driving wheel and the actuator.
